(19) United States
(12) Patent Application Publication
(10) Pub. No.: US 2010/0303752 A1
(43) Pub. Date: Dec. 2, 2010

(54) USE OF POLYMERIC GUANIDINES FOR CONTROLLING MICROORGANISMS

(75) Inventors: Christa Hametner, Wien (AT); Oskar Schmidt, Wien (AT)

Correspondence Address: Workman Nydegger;1000 Eagle Gate Tower, 60 East South Temple, Salt Lake City, UT, 84111, US

(73) Assignee: AKA TECHNOLOGY GMBH, Wien (AT)

(21) Appl. No.: 12/523,845

(22) PCT Filed: Dec. 28, 2007
(86) PCT No.: PCT/AT07/00589

§ 371 Date: Apr. 14, 2010

(30) Foreign Application Priority Data
Dec. 29, 2006 (AT) A 2156/2006
Dec. 19, 2007 (EP) 07150147.2

(51) Int. Cl. A01N 47/44; A01P 1/00; C08G 73/00; A61K 8/43; C09K 5/00

(52) U.S. Cl. 424/78.37; 252/68; 528/422; 514/772.3

(57) ABSTRACT

The invention relates to the use of polymeric guanidinium hydroxides based on a diamine which contains oxyalkylene chains and/or alkylene groups between two amino groups, obtainable by polycondensation of a guanidine acid addition salt with the diamine, whereby a polycondensation product in the form of a salt is obtained, which subsequently is converted into the hydroxide form via basic anion exchange, for controlling microorganisms.

The present invention relates to the use of polymeric guanidines for controlling microorganisms.

Biocidal polymers based on guanidinium hydrochloride, in particular their effect against Escherichia coli bacteria, are already known (cf. WO 01/85676). Furthermore, it is already known that such guanidine derivatives can be used as fungicidal agents (cf. WO 2006/047800). The polymers Akacid®, poly-[2-(2-ethoxy)-ethoxyethyl guanidinium chloride], and Akacid Plus®, a 3:1 mixture of poly-(hexamethylene guanidinium chloride) and poly-[2-(2-ethoxy)-ethoxyethyl)-guanidinium chloride] are of special importance (cf. Antibiotika Monitor, 22th volume, issue Jan. 2, 2006, online edition under http://www.antibiotikamonitor.at/06_{—}12/06_{—}12 inhalt.htm).

Since the ecological and economical demands on modern biocidal agents are increasing continuously, for example, with regard to the spectrum of activities, the toxicity, the selectivity, the amount of expenditure, the residue formation and a favourable producibility, and, moreover, for example problems associated with resistances may arise, there is the constant challenge of developing new agents.

In addition, the universal use for room and surface disinfection requires agents which should exhibit several properties: they must have a microbicidal activity which is as strong as possible and must be applicable by the atomization technique, they must not attack or corrode, respectively, the environment such as, e.g., electronic devices, but should not have a toxic effect on mammals and in particular humans, and, moreover, they should also be environmentally compatible. To date, no agent has been known which meets all those demands simultaneously and to a satisfactory extent.

The present invention provides a use which, at least in partial aspects, solves the problem posed, since it has now surprisingly been found that polymeric guanidinium hydroxides can be used particularly well for controlling undesirable microorganisms such as bacteria, yeasts and fungi.

A preferred embodiment of the guanidinium hydroxides usable according to the invention is based on a diamine which contains oxyalkylene chains and/or alkylene groups between two amino groups and is obtainable particularly preferably by polycondensation of a guanidine acid addition salt with the diamine, whereby a polycondensation product in the form of a salt is obtained, which subsequently is converted into the hydroxide form via basic anion exchange.

The salts usable as starting materials for said embodiment of the guanidinium hydroxides usable according to the invention, in particular hydrochlorides, are known (cf. WO 01/85676). For example, a further preferred embodiment of the polymeric guanidinium hydroxide usable according to the invention, poly-[2-(2-ethoxy-ethoxyethyl)-guanidinium hydroxide] having an average molecular mass of about 1000, is obtained by dissolving 4.43 moles of guanidinium hydrochloride in 4.03 moles of triethylene glycol diamine at 50° C. Subsequently, this is heated to 120° C. and stirred for 2 hours at said temperature. Thereupon, the temperature is maintained for 2 hours, then a vacuum (0.1 bar) is applied and stirring is continued under vacuum for 2 more hours at 170° C. Subsequently, this is aerated to normal pressure, allowed to cool to 120° C. and diluted with demineralized water to approx. 50%. It is neutralized to a pH value of approx. 6 with phosphoric acid, allowed to cool and diluted to the desired concentration. Subsequently, the hydroxide form is produced by treating the solution obtained previously with a strongly alkaline anion exchanger in a hydroxide form, suitably in an exchange column (e.g. “Ambersep 900 OH” or “Lewatit MP 500”). In this manner, the guanidinium hydroxide usable according to the invention is obtained which has the following characteristics: empirical formula: C₂₁H₅₁N₉O₅ or, respectively, 49.5% carbon, 10% hydrogen, 24.8% nitrogen and 15.7% oxygen.

A further preferred embodiment of the guanidinium hydroxide usable according to the invention consists in that, among the representatives of the family of polyoxyalkylene guanidine salts, such using triethylene glycol diamine (relative molecular mass: 148), polyoxypropylene diamine (relative molecular mass: 230) as well as polyoxyethylene diamine (relative molecular mass: 600) or also polyhexamethylene diamine (relative molecular mass:) are obtained.

A further preferred embodiment of the guanidinium hydroxide usable according to the invention is characterized in that poly-[2-(2-ethoxy-ethoxyethyl)-guanidinium hydroxide] comprising at least 3 guanidinium groups is contained as the polymeric guanidine derivative.

Furthermore, guanidinium hydroxides can be used preferably in which the polycondensation product in salt form can be obtained beforehand by polycondensation of a guanidine acid addition salt with an alkylene diamine and an oxyalkylene diamine at a molar ratio ranging between 4:1 and 1:4 (alkylene diamine/oxyalkylene diamine), preferably between 3:1 and 1:3.

An alkylene diamine of general formula

H₂N—(CH₂)ₙ—NH₂

is preferably provided as the diamine, wherein n is an integer between 2 and 10, in particular 6.

Furthermore, a compound of general formula

H₂N—[CH₂)₂O)]ₘ—(CH₂)₂—NH₂

is preferably provided as the oxyalkylene diamine, wherein m is an integer between 2 and 5, in particular 2.

The average molecular mass of the guanidinium hydroxide usable according to the invention preferably ranges from 500 to 3,000, particularly preferably from 500 to 2,000, exceptionally preferably from 500 to 1,500. Furthermore, an average molecular mass of 1000 is preferred.

Preferred uses of the polymeric guanidinium hydroxide are
as an atomizing agent for microbial decontamination;
decontamination of building structures;
as a preservative for washing and cleaning agents;
as a preservative for paints and lacquers;
as a preservative for cosmetics;
as an antimicrobial additive in natural and synthetic polymers and leather, wherein the synthetic polymers are preferably formed as fibres or films or as coating materials;
as an anti-slime agent in paper manufacture and petroleum production; and
as an additive for cooling lubricants.

Cooling lubricants are needed for the machining of metals. It is understood that “machining” means all mechanical working methods in which the metal is brought into the desired shape by removing it in the form of cuttings. For example, turning, drilling, milling and grinding belong to machining techniques. Those activities are usually performed using a lathe or a drill.

It is the function of the cooling lubricant to reduce the friction between the metallic workpiece and the tool, whereby the wear of the tool is decreased, and also to resist the heating of the material. At temperatures above 1000° C., cooling lubricants allow higher speeds of operation. However, the cooling lubricant can also serve for flushing the cuttings from the working sphere and protecting the metallic workpiece from corrosion.

Various cooling lubricants are known in the prior art. There are, for example, mineral oils with or without additives, which are not miscible with water, wherein those additives may be fatty acids or sulfidic and phosphoric organic compounds. Furthermore, milky white oil-in-water emulsions containing mineral oils are known. On the other hand, solutions free from mineral oils are transparent and contain sodium carbonate or nitrite in water.

Substances which provide an additional advantage with their chemical properties, such as, e.g., rust protection, a higher lubricating effect under extreme conditions, a prevention of foam formation or a slowing down of the growth of bacteria, yeasts and fungi in the emulsion, serve as additives.

The disposal of spent cooling lubricants is expensive so that the trend is toward cleaning the agents after use and reusing them multiple times. However, those cooling lubricants which have been used multiple times are particularly susceptible to microorganisms. If the number of germs is too high, the solution might “tip over”.

The guanidinium hydroxide used is preferably poly-[2-(2-ethoxy-ethoxyethyl)-guanidinium hydroxide] having an average molecular mass of about 1000 (A-1), an average molecular mass of about 1500 (A-2), an average molecular mass of about 500 (A-3), an average molecular mass of about 2000 (A-4), an average molecular mass of about 2500 (A-5) or an average molecular mass of about 3000 (A-6) as well as furthermore a polycondensate from poly-(hexamethylene guanidinium hydroxide) and poly-[2-(2-ethoxy)-ethoxyethyl)-guanidinium hydroxide] at a molar ratio of 3:1 (A-7).

The agents according to the invention can be used as such or depending on their respective physical and/or chemical properties in the form of their formulations or the application forms produced therefrom, such as aerosols, capsule suspensions, cold fog concentrates, hot fog concentrates, encapsulated granules, fine granules, pourable concentrates for the treatment of microbially contaminated materials, ready-to-use solutions, atomizable powders, emulsifiable concentrates, oil-in-water emulsions, water-in-oil emulsions, macrogranules, microgranules, powders dispersible in oil, pourable concentrates miscible with oil, liquids miscible with oil, foams, pastes, seeds enveloped by pesticides, suspension concentrates, suspension-emulsion concentrates, soluble concentrates, suspensions, wettable powders, soluble powders, dusting agents and granules, water-soluble granules or tablets, water-soluble powders and wettable powder formulations.

### EXAMPLES

Below, the excellent properties of the guanidine hydroxide usable according to the invention are described in further detail.

### Example 1

This example is supposed to document the superiority of the OH form of the polymeric guanidine relative to the chloride form.

TABLE
Analyses of the effectiveness of the OH form

| | MHK (mg/l) | | | | |
|---|---|---|---|---|---|
| | S. aureus | E. coli | P. aeruginosa | C. albicans | S. cerevisiae |
| Concentration | ATCC 6538 | ATCC 10536 | ATCC 15422 | ATCC 10231 | LS 17 |
| Solution 10% | 1 | 2 | 32 | 32 | 4 |
| Solution 8% | 0.5 | 1 | 16 | 16 | 4 |
| Solution 25% | 0.25 | 1 | 8 | 8 | 4 |

MHK: minimum inhibition concentration, determined by the microdilution method

TABLE
Analyses of the effectiveness of the Cl form

| | MHK (mg/l) | | | | |
|---|---|---|---|---|---|
| | S. aureus | E. coli | P. aeruginosa | C. albicans | S. cerevisiae |
| | ATCC 6538 | ATCC 10536 | ATCC 15422 | ATCC 10231 | LS 17 |
| Solution 25% | 2-8 | 1-2 | 16-32 | 16-32 | 8 |

By replacing the chloride ions with hydroxide, a significantly larger field of application for guanidine derivatives is thus opened up, since a chloride-induced pitting corrosion of metals is prevented especially if those derivatives are regularly applied as preservatives, protectants or disinfectants. Likewise, their use as additives for cooling lubricants especially in the processing of workpieces made of aluminium is possible only in the hydroxide form.

In addition, the environmental compatibility of the products is significantly improved by said modification, since chloride in the groundwater increases the solubility of heavy metals, salinizes soils and forms hydrochloric acid in case of a thermal disposal and in case of fire, respectively.

### Example 2

The activity as a preservative was tested, for example, in a mineral inner wall paint by adding 0.2 to 2% by weight of guanidine derivative with germ-infested tap water, wherein a complete germ destruction is detectable from 1%. For this purpose, the paint samples were diluted 1:10 (volume part paint: volume part water) with germ-infested tap water. 100 μl of said dilution was, in each case, spread on TSA plates (bacterial growth medium), incubated at 35° C. for 24 hours, and subsequently the total germ number was determined in the form of colony-forming units (CFU).

TABLE

| | CFU/100 μl paint |
|---|---|
| Sample description | water mixture* |
| Unpreserved paint | 41 |
| Paint with 1% guanidine derivative, dispersed via | 7.5 |
| pigment paste | |
| Paint with 2% guanidine derivative, dispersed via | 0 |
| pigment paste | |
| Paint with 0.2% guanidine derivative, added via | 33.5 |
| V-solution | |
| Paint with 1% guanidine derivative, added via | 0 |
| V-solution | |
| Paint with 2% guanidine derivative, added via | 0 |
| V-solution | |
| Conventionally preserved paint | 0 |

*CFU: colony-forming units, arithmetic mean value of two determinations

### Example 3

Based on their good skin and mucous membrane tolerance (non-irritant on the skin and in the eyes, no sensitization), the guanidine derivatives forming the subject matter are also suitable as preservatives for cosmetics and even as disinfectants for direct application on the skin.

TABLE

| Study | Result |
|---|---|
| Acute oral toxicity LD 50 oral, rat, | >2000 mg/kg |
| OECD 423 | |
| Acute dermal toxicity LD 50 dermal, | >2000 mg/kg |
| OECD 402 | |
| Acute irritation of the skin, OECD 404 | non-irritant |
| Acute irritation of the eyes, OECD 405 | non-irritant |
| Sensitization: Local lymph node assay | non-sensitizing for the skin |

### Example 4

The polymeric guanidine hydroxides usable according to the invention are soluble in water and can be applied with conventional atomizing devices in the form of ultra-fine droplets, virtually as a fog of active agents, in closed rooms for the disinfection of ambient air and surfaces.

Said atomizing application permits quick control of bacteria, fungi and viruses. All contaminated spaces such as mould-infested residences, air conditioning systems, germ-infested food production plants, areas for livestock husbandry and also public facilities (hospitals, spa areas, schools) can be treated quickly and effectively. For an effective treatment, the spaces to be treated should be closed.

The atomizing application with the polymeric guanidine hydroxides according to the invention has the following advantages over conventional wiping and spraying applications:
(a) Not only are contaminated surfaces covered completely, but also the ambient air itself is decontaminated.
(b) The active agent eliminates not only pathogens, but also unpleasant smells located in the air.
(c) The contained water is in fog form also an ideal medium for transporting the active agent into cracks, joints and gaps, which are otherwise difficult to treat.
(d) A quick, automatable and controllable application
(e) High effectiveness despite low use-levels
   The small droplet sizes in the μm range permit a quick evaporation of the water and thus a concentration of the active agent on the surface of particles and germs.
(f) The dry fog prevents harmful moisture (electrical equipment, moisture corrosion)
(g) During the atomizing phase, the active agent adheres to all particles and surfaces and walls, respectively, and thus also forms a preventive, permanently effective protective film.

Thus, the invention also concerns a decontamination by atomization of the polymeric guanidine derivative in residences, air conditioning systems and ventilation shafts, hotels and catering areas, food and pharmaceutical production plants, storage and transport devices, particularly ships and cars, areas for livestock husbandry, laboratories and also public facilities such as hospitals, spa areas, schools.

The amount of active agents which is used is optimized to a required minimum. The procedure and the type of the atomizing devices are adjusted to the functional range and the room size.

On average, 0.025 litres to at most 0.075 litres per m³ room volume of a 0.08 to 2% (by weight) solution containing guanidine derivative are emitted.

The efficiency and effect of said atomizing application have been tested, for example, in the fermenting cellar and laboratory of a brewery. For this purpose, samples of surfaces were taken with a swab before and after the treatment and spread on culture media and evaluated. In the table, (−) means no growth, (+) a very low germ content, + a low germ content, ++ an average germ content, +++ a high germ content, ++++ a very high germ content, R bacteria bed growth, nn non-detected.

A clear reduction in the total germ number (bacteria, enterobacteria, yeasts and moulds) could be detected.

TABLE
laboratory

| | Result of the examination | | | | | | | |
|---|---|---|---|---|---|---|---|---|
| | total germ | | entero- | | | | | |
| | number | | bacteria | | yeasts | | moulds | |
| Sampling point | before | after | before | after | before | after | before | after |
| Light switch | + | − | − | − | − | − | − | − |
| Output of the air | + | − | + | − | − | − | − | − |
| conditioning system | | | | | | | | |
| Left side wall | + | − | − | − | + | − | + | − |
| Window sill | + | − | − | − | ++ | − | − | − |
| Baseboard below the | ++ | + | − | − | − | − | − | − |
| window | | | | | | | | |
| Light switch at the | ++ | − | − | − | − | − | − | − |
| window in the centre | | | | | | | | |
| Water tap | ++ | − | − | − | + | − | − | − |
| Wooden cupboard on the | R | − | R | − | + | − | − | − |
| right | | | | | | | | |
| Joints on the tile table | R | + | R | + | ++ | − | + | − |
| Ventilation shaft in the | ++ | − | − | − | − | − | + | − |
| rear area of the laboratory | | | | | | | | |
| Steel rail over the last | +++ | − | − | − | − | − | +++ | − |
| window | | | | | | | | |

TABLE
Fermenting cellar

| | Result of the examination | | | | | | | |
|---|---|---|---|---|---|---|---|---|
| | total germ | | entero- | | | | | |
| | number | | bacteria | | yeasts | | moulds | |
| Sampling point | before | after | before | after | before | after | before | after |
| Stair wall below the | ++ | − | − | − | ++ | − | + | − |
| stairs | | | | | | | | |
| Cable channel of the stair | − | − | − | − | − | − | − | − |
| wall | | | | | | | | |
| Mould field above the | − | − | − | − | − | − | ++ | − |
| cable | | | | | | | | |
| Ventilation shaft | − | − | − | − | − | − | + | − |
| Counterwall on the | R | − | R | − | ++ | − | ++ | − |
| moulding | | | | | | | | |
| Door lintel counterwall | +++ | ++ | +++ | − | ++ | − | +++ | − |
| Right wall beside the | ++ | − | ++ | − | + | − | − | − |
| door | | | | | | | | |
| Right wall beside the | +++ | + | ++ | + | + | − | − | − |
| column | | | | | | | | |
| Stainless steel sheet | R | + | R | − | R | − | ++ | − |
| (centre) | | | | | | | | |
| Valve column in the | R | ++ | R | + | ++ | − | ++ | − |
| middle of the room | | | | | | | | |
| Joints on the electrical | R | ++ | R | + | +++ | − | + | − |
| cabinet | | | | | | | | |
| Tank 202 | R | + | R | + | − | − | + | − |
| Upper aisle joints on the | R | ++ | +++ | − | − | − | − | − |
| water tap | | | | | | | | |
| Electric motor in the | R | ++ | +++ | + | +++ | −− | + | − |
| upper aisle | | | | | | | | |
| Socket in the upper aisle | R | ++ | R | + | ++ | | − | − |

1. Use of polymeric guanidinium hydroxides for controlling microorganisms.

2. Use according to claim 1, wherein the polymeric guanidinium hydroxides is based on a diamine which contains oxyalkylene chains and/or alkylene groups between two amino groups.

3. Use according to claim 2, wherein the polymeric guanidinium hydroxides is obtainable by polycondensation of a guanidine acid addition salt with the diamine, whereby a polycondensation product in the form of a salt is obtained, which subsequently is converted into the hydroxide form via basic anion exchange.

4. Use according to any of claims 1 to 3, wherein the polymeric guanidinium hydroxide is poly-[2-(2-ethoxy-ethoxyethyl)-guanidinium hydroxide] comprising at least 3 guanidinium groups.

5. Use according to claim 3, wherein the polycondensation product in salt form is obtainable by polycondensation of a guanidine acid addition salt with an alkylene diamine and an oxyalkylene diamine at a molar ratio ranging between 4:1 and 1:4 (alkylene diamine/oxyalkylene diamine).

6. Use according to claim 2, wherein an alkylene diamine of general formula
H₂N—(CH₂)ₙ—NH₂
is provided as the diamine, wherein n is an integer between 2 and 10, in particular 6.

7. Use according to claim 2, wherein a compound of general formula
H₂N—[CH₂)₂O)]ₘ—(CH₂)₂—NH₂
is provided as the oxyalkylene diamine, wherein m is an integer between 2 and 5, in particular 2.

8. Use according to claim 2, wherein the average molecular mass of the polymeric guanidinium hydroxide ranges from 500 to 3,000.

9. Use of a polymeric guanidinium hydroxide according to claim 2, wherein the polymeric guanidinium is used as an additive for cooling lubricants.

10. A cooling lubricant for the machining of metals containing an additive, wherein a polymeric guanidinium hydroxide as defined in claims 2 is provided as the additive.

11. Use of a polymeric guanidinium hydroxide according to claim 2, wherein the polymeric guanidinium is used as an atomizing agent for microbial decontamination.

12. Use according to claim 11, wherein the microbial decontamination comprises the decontamination of building structures.

13. Use of a polymeric guanidinium hydroxide according to claim 2, wherein the polymeric guanidinium is used as a preservative for washing and cleaning agents.

14. Use of a polymeric guanidinium hydroxide according to claim 2, wherein the polymeric guanidinium is used as a preservative for paints and lacquers.

15. Use of a polymeric guanidinium hydroxide according to claim 2, wherein the polymeric guanidinium is used as a preservative for cosmetics.

16. Use of a polymeric guanidinium hydroxide according to claim 2, wherein the polymeric guanidinium is used as an antimicrobial additive in natural and synthetic polymers and leather.

17. Use according to claim 16, wherein the synthetic polymers are formed as fibres or films or as coating materials.

18. Use of a polymeric guanidinium hydroxide according to claim 2, wherein the polymeric guanidinium is used as an anti-slime agent in paper manufacture and petroleum production.

19. Use of a polymeric guanidinium hydroxide according to claim 2, wherein the polymeric guanidinium is used as an atomizing agent for microbial decontamination.
